(19) United States
(12) Patent Application Publication
(10) Pub. No.: US 2011/0064680 A1
(43) Pub. Date: Mar. 17, 2011

(54) NATURALLY DERIVED WATERPROOFING AGENTS FOR AQUEOUS-BASED PERSONAL CARE PRODUCTS

(75) Inventors: Michael B. Davies, Dacula, GA (US); Norman L. Kennedy, Darien, GA (US); Kerry L. Thompson, Savannah, GA (US)

(73) Assignee: Pinova, Inc., Brunswick, GA (US)

(21) Appl. No.: 12/878,219

(22) Filed: Sep. 9, 2010

Related U.S. Application Data
(60) Provisional application No. 61/241,447, filed on Sep. 11, 2009.

(51) Int. Cl. A61K 8/72; A61Q 17/04

(52) U.S. Cl. 424/59

(57) ABSTRACT

Personal care compositions such as sunscreens, color make-up, lotions and creams which contain an amount of a hydrogenated rosin or resin which provides increased resistance to removal by water of such personal care compositions.

### FIELD OF THE INVENTION

This invention is related to personal care compositions such as sunscreens, color make-up, lotions and creams in general and to personal care compositions which are capable of providing a benefit to the body over an extended period of time by increasing the resistance to removal by water of such personal care compositions.

### BACKGROUND OF THE INVENTION

There are many cases where the desire is for a personal care formula to be applied to the skin and to remain on the skin for as long as possible. The final product should also be aesthetically pleasing to the customer with no irritation or negative attributes. One of the primary reasons for unintentional removal of a product from the skin is water. Rain, sweat and tears are a few of the forms that water can take that can cause product to be removed from the skin. This can be difficult since heavy, synthetic polymers are the most commonly used materials at this time.

U.S. Pat. No. 4,810,490, which is incorporated herein by reference in its entirety, is directed to sunscreen compositions which when applied to human skin provide protection against erythema caused by ultraviolet radiation from sunlight. The object of this patent was to provide sunscreen compositions capable of protecting the skin against erythema over prolonged periods of time. The compositions of the patent comprise a resin which is primarily the hydrogenated methyl ester of wood rosin; an ultraviolet sun-screening agent; an emollient and optionally, the compositions may include small but effective amounts of opacifiers, surfactants, fragrances and other compatible agents to obtain desired cosmetic aesthetics. In order to produce the compositions of the patent, the hydrogenated

There is a need for the provision a personal care composition which exhibits increased resistance to removal by water and avoids the use of heavy, synthetic polymers or organic solvents but rather, and in view of the growing desire for natural based products, uses rosin or polyterpene resins which add the benefit of being derived from a renewable resource and uses aqueous solvents, which avoids the use of organic solvents.

### BRIEF DESCRIPTION OF THE FIGURES

FIG. 1 is a micrograph at 40× magnification of VP/Eicosene Copolymer prototype neat after one year at room temperature.

FIG. 2 is a micrograph at 40× magnification hydrogenated methyl abietate emulsion at room temperature.

### SUMMARY OF THE INVENTION

The present invention relates to a personal care composition comprising: about 0.1% to about 10.0% by weight of hydrogenated rosin, and its derivatives and/or selected polyterpene resins. The hydrogenated rosin derivatives and/or polyterpene resins are combined with sunscreen agents, water and optionally effective amounts of emollients, opacifiers, fragrances, surfactants, preservatives and other desirable and compatible agents. The resultant composition is a personal care composition with increased resistance to removal by water.

### DETAILED DESCRIPTION OF THE INVENTION

The present invention relates to the use of hydrogenated rosin and derivatives and/or polyterpene resins in sunscreens as waterproofing agents. In addition to imparting improved resistance to removal by water, the hydrogenated rosin and/or polyterpene resin can be used as a solubilization agent for solid sunscreen agents. Waterproof sunscreen tests were run and solubility profiles were

The hydrogenated rosin or resin derivatives of use in the present invention may be based on either a hydrogenated wood rosin or a hydrogenated gum rosin, where wood rosin is obtained from the extraction of rosin from pine stumps while the gum rosin is obtained from the extraction of rosin from the tapped trees. Preferred rosin resins are esters of hydrogenated rosin based on monoalcohols such as methanol, ethanol or the propanols, or diols such as diethylene glycol, triethylene glycol and propylene glycol, or triols such as glycerol and tetraols such as pentaerythritol. Especially preferred resins are methyl esters an example being Hercolyn® D methyl ester of hydrogenated rosin, a highly viscous liquid resin, and glycerol esters, an example being Endere™ S glycerol ester of highly hydrogenated rosin. Both rosin esters are available from Ashland Inc. It will be recognized by those familiar with the art of rosin chemistry that mixtures of various rosin ester grades can be prepared to give suitable performance as waterproofing agents.

Polyterpene resins are thermoplastic resins that are produced by the catalyzed polymerization of terpenes, in a suitable reaction solvent. Examples of terpenes used to manufacture polyterpene resins are alpha-pinene, beta-pinene, dipentene (racemic limonene), myrcene, delta-3-carene and numerous menthadienes such as alpha and beta phellandrenes, alpha and gamma terpinenes and terpinolene, and mixtures of these terpenes. The aforementioned terpenes are all derived from turpentines obtained by the pulping of pine trees to make paper, or from the solvent extraction of pine tree stumps (called wood turpentine) or from the tapping of pine trees (called gum turpentine). Alpha pinene, beta pinene, delta-3-carene, and delta-2-carene can be pyrolized to give mixtures of terpenes that can be used in part or with other terpene streams to produce polyterpene resins.

Another important terpene used to manufacture polyterpene resins is d-limonene, which is available from citrus processing. Catalysts that are used or can

The rosins or resins is present in the personal care composition, more particularly in the sunscreen of the present invention in amounts ranging from about 0.1% to about 10.0%, preferably about 0.5% to about 4.0%, still more preferably from about 1.0 to about 3.0% by weight of the personal care composition.

While not wishing to be bound by theory, it is believed that the chemical and physical properties of the hydrogenated rosin or resin (s) improves the water resistance of the sunscreen by forming a substantially continuous hydrophobic film, and exhibits a higher degree of adhesion to skin. These resinous products can be easily solubilized in oil based products, or emulsified for incorporation into water based screens and lotions.

The personal care composition of the present invention also may comprise an emollient material, wherein such emollient material may be selected from among hydrocarbon oils and waxes, as well as fatty acid esters of low molecular weight alcohols such as butyl stearate, isopropyl stearate, isopropyl palmitate, isopropyl myristate and volatile silicone fluids. The emollient material is present in amounts typically used in the formulation of such personal care compositions and range from about 5.0% to about 60.0% by weight of the total personal care composition.

If present in the compositions of this invention may include small but effective amounts of opacifiers, fragrances, surfactants, preservatives and other desirable and compatible agents to obtain desired personal care aesthetics.

Among the advantages of the use of the hydrogenated rosin derivatives or polyterpene resins, is the ability to formulate personal care compositions without the addition of an organic solvent, such as an aliphatic hydrocarbon, ether, ester,

Another advantage of using hydrogenated rosin or resins is that they have been found to directly solubilize solid sunscreen agents without the need for the addition of an organic solvent.

The personal care compositions of the present invention may be formulated by simply admixing all of the components. However, it may be advantageous to first combine the solid sunscreen agent with the hydrogenated rosin or resins to dissolve the solid sunscreen agent in the hydrogenated rosin or resins prior to combining the sunscreen agent dissolved in the hydrogenated rosin or resins with the other components of the personal care composition.

The use of the hydrogenated rosin or resins should impart similar waterproofing results for other personal care products such as color cosmetics, moisturizers, topical analgesics, creams and the like.

The present invention is further illustrated in the following examples, wherein all parts or percentages mentioned are by weight unless otherwise indicated. These examples are given only by way of illustration and are not intended to limit the invention except as set forth in the claims.

### EXAMPLES

### Waterproof Sunscreen with an Estimated SPF 15

| | |
|---|---|
| Avobenzone | 2.00% by weight |
| Octocrylene | 1.50% by weight |
| Octisalate | 5.00% by weight |
| Oxybenzone | 3.00% by weight |
| Hydrogenated Methyl Abietate | 2.00% by weight |
| Dimethyl Capramide | 2.00% by weight |
| Acrylates/C10-30 Alkyl Acrylate Crosspolymer | 0.35% by weight |
| Preservative | 0.70% by weight |
| Water | 83.10% by weight |
| Triethanolamine | 0.35% by weight |

During the making of the sunscreens, it was observed that the systems containing the hydrogenated wood resins more easily solubilzed the solid sunscreens (Oxybenzone and Avobenzone). This result was unexpected so additional work was done to determine the solubility of these actives in the hydrogenated wood resins. The results were:

| | |
|---|---|
| Avobenzone | 13% |
| Oxybenzone | 24% |

To address how well the hydrogenated wood resins work as waterproofing agents in sunscreen lotions three prototype formulas were made up. The only difference between the formulas was the waterproofing agent used; the level of these materials was consistent between all the formulas. The water proofing agents were an industry standard (Ganex V-220; VP/Eicosene copolymer, available from International Specialty Products), a solid hydrogenated wood resin and a liquid wood resin.

| | |
|---|---|
| Avobenzone | 2.00% by weight |
| Octocrylene | 1.50% by weight |
| Octisalate | 5.00% by weight |
| Oxybenzone | 3.00% by weight |
| Waterproofing Agent | 2.00% by weight |
| Dimethyl Capramide | 2.00% by weight |
| Acrylates/C10-30 Alkyl Acrylate Crosspolymer | 0.35% by weight |
| Preservative | 0.70% by weight |
| Water | 83.10% by weight |
| Triethanolamine | 0.35% by weight |

The waterproofing test was run using the method defined in the FDA Sunscreen Monograph. Five subjects were evaluated in each case. The results were:

| | Waterproof Results |
|---|---|
| Ganex V-220 | 16.20 |
| Liquid Resin | 16.20 |
| Solid Resin | 16.80 |

The results show that the hydrogenated wood resins are comparable to the industry standard as far as waterproofing sunscreens.

Micrographs were taken of formulations containing either the Ganex V-220; VP/Eicosene copolymer, or the liquid resin, both at room temperature and at a 40× magnification.

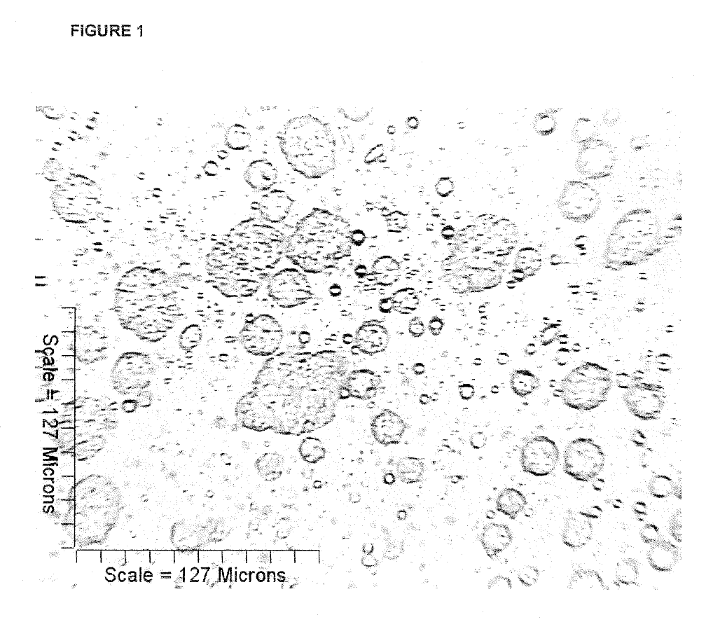

In FIG. 1, an image of the VP/Eicosene Copolymer prototype neat after one year at room temperature is provided. It is noted that the shape and size of the particles of the copolymer and their granular surface texture.

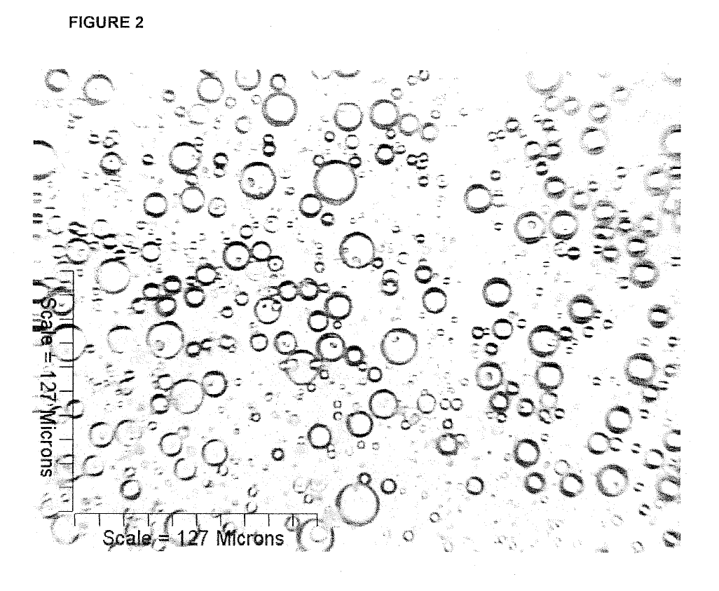

In FIG. 2, an image of a liquid version of hydrogenated methyl abietate is provided. In the visual comparison of the image of FIG. 1 and of FIG. 2, it appeared that the abietate formula had a more stable emulsion because of the

Although the invention has been described with referenced to preferred embodiments, it is to be understood that variations and modifications in form and detail thereof may be made without departing from the spirit and scope of the claimed invention. Such variations and modifications are to be considered within the purview and scope of the claims appended hereto.

What is claimed:

1. A personal care composition comprising: about 0.1% to about 10.0% by weight of hydrogenated rosin or resin derivative, or a polyterpene resin, a sunscreen agent, water and optionally effective amounts of emollients, opacifiers, fragrances, surfactants, preservatives and other desirable and compatible agents.

2. A process for making a personal care composition comprising:
a Obtaining a hydrogenated rosin or resin;
b. Dissolving an amount of a sunscreen agent in the hydrogenated rosin or resin to produce a rosin or resin solvated sunscreen agent; and
c. admixing the rosin or resin solvated sunscreen agent with water and at least one of, emollients, ° pacifiers, fragrances, surfactants, preservatives and other desirable and compatible agents to produce a personal care composition.

3. A composition according to claim 1 where the rosin resin is based on hydrogenated wood rosin or hydrogenated gum rosin.

4. A composition according to claim 1 where the rosin resin is an ester, or mixtures of esters of two or more alcohols or polyols of hydrogenated wood rosin or hydrogenated gum rosin.

5. A composition according to claim 1 where the rosin resin is an ester based on methanol, diethylene glycol, triethylene glycol, propylene glycol, glycerol or pentaerythritol.

6. A composition according to claim 1 where the polyterpene resin is based on d-limonene from citrus processing.

7. A composition according to claim 1 where the polyterpene resin is based on limonene mixed with either alpha-pinene, beta-pinene, delta-3-carene, myrcene, alpha phellandrene, beta phellandrene, alpha-terpinene, gamma-terpinene, terpinolene or the pyrolysis products of alpha pinene or beta pinene or delta-3-carene or delta-2-carene or mixtures thereof.
